(19) United States
(12) Patent Application Publication
(10) Pub. No.: US 2019/0060203 A1
(43) Pub. Date: Feb. 28, 2019

(54) HAIR SHAPING COMPOSITION

(71) Applicant: Conopco, Inc., d/b/a UNILEVER, Englewood Cliffs, NJ (US)

(72) Inventors: Andrew Malcolm MURRAY, Neston (GB); Prem Kumar Cheyalazhag PAUL, Spital, Wirral (GB); Susan PYE, Wirral (GB); Janhavi Sanjay RAUT, Bangalore (IN)

(73) Assignee: Conopco, Inc., d/b/a UNILEVER, Englewood Cliffs, NJ (US)

(21) Appl. No.: 16/075,845

(22) PCT Filed: Jan. 6, 2017
(86) PCT No.: PCT/EP2017/050256

§ 371 Date: Aug. 6, 2018

(30) Foreign Application Priority Data
Feb. 11, 2016 (EP) 16155220.3

(51) Int. Cl. A61K 8/64; A61Q 5/06; A61K 8/365; A45D 7/04; A45D 7/06

(52) U.S. Cl. CPC A61K 8/64; A61Q 5/06; A45D 7/06; A45D 7/04; A61K 8/365

(57) ABSTRACT

The invention provides a hair shaping composition suitable for topical application to hair, the composition having a pH of 6 or less and comprising, in an aqueous continuous phase: (i) at least 0.5% (by weight based on the total weight of the composition) of one or more silk sericins, and (ii) at least 0.5% (by weight based on the total weight of the composition) of one or more aliphatic di- or tricarboxylic acids or salts or hydrates thereof. 15 The invention also provides a method for shaping hair which comprises the steps of (a) treating the hair by topical application of a hair shaping composition as defined above, followed by (b) mechanically shaping the treated hair.

### FIELD OF THE INVENTION

This invention relates to a hair shaping composition, and more particularly a hair shaping composition which does not require the breakage of hair disulfide bonds.

### BACKGROUND AND PRIOR ART

Many people with naturally kinky, curly, or even wavy hair often desire to straighten their hair. Permanent hair straightening compositions that are on the market are based on chemical treatment of the hair in a two-step process using reducing agents to break hair disulfide bonds, followed by a neutralisation or oxidation step to re-establish new disulfide bonds in the desired configuration. Such systems have various negatives associated with them; in that the process itself is difficult to conduct, in many instances this straightening process is undertaken by a qualified hairdresser in a professional salon. Furthermore the straightening process damages the hair, has an unpleasant odour and can cause irritation to the scalp.

Surprisingly we have found that hair can be shaped without causing the chemical damage which is traditionally associated with permanent hair straightening processes involving breakage of the hair disulfide bonds.

Advantageously the method of the invention can be accomplished by a consumer without intervention of a professional hairdresser. Furthermore, hair shaped with the method of the invention remains shaped even after subsequent washing.

### SUMMARY OF THE INVENTION

The present invention provides a hair shaping composition suitable for topical application to hair, the composition having a pH of 6 or less and comprising, in an aqueous continuous phase:

(i) at least 0.5% (by weight based on the total weight of the composition) of one or more silk sericins, and

(ii) at least 0.5% (by weight based on the total weight of the composition) of one or more aliphatic di- or tricarboxylic acids or salts or hydrates thereof.

The invention also provides a method for shaping hair which comprises the steps of (a) treating the hair by topical application of a hair shaping composition as defined above, followed by (b) mechanically shaping the treated hair.

### DETAILED DESCRIPTION AND PREFERRED EMBODIMENTS

All molecular weights as used herein are weight average molecular weights, unless otherwise specified.

By “aqueous continuous phase” is meant a continuous phase which has water as its basis.

A hair shaping composition according to the invention will generally comprise at least 60%, preferably at least 70% and more preferably at least 80% water (by weight based on the total weight of the composition). Preferably, the composition comprises no more than 99% and more preferably no more than 98% water (by weight based on the total weight of the composition). Other organic solvents may also be present, such as lower alkyl alcohols and polyhydric alcohols. Examples of lower alkyl alcohols include C₁ to C₆ monohydric alcohols such as ethanol and isopropanol. Examples of polyhydric alcohols include propylene glycol, hexylene glycol, glycerin, and propanediol. Mixtures of any of the above described organic solvents may also be used.

The hair shaping composition of the invention comprises, as component (i), one or more silk sericins.

Silks can be broadly defined as externally spun fibrous protein secretions, made by arthropods for a variety of task-specific applications. Silk fibres are typically composite materials formed of silk protein and other associated molecules such as glycoproteins and lipids.

Silkworms produce silk cocoons to protect themselves during their metamorphosis into moths, and humans have harvested silk fibres from these cocoons for centuries to produce textiles. Of all natural silk-producing animals, mulberry silkworms (Bombyx mori) are of the most economic importance, because it is possible to rear them in captivity. Other than the domesticated B. mori, silk fibre production is reported from the wild non-mulberry saturniid variety of silkworms, such as tasar (Antheraea mylitta), muga (Antheraea assamensis) and en (Philosamia ricini).

Silkworm (e.g. B. mori) silks are composed of two groups of proteins. The fibroins, which constitute the silk thread, are synthesized in the posterior part of the silk gland (PSG). The sericins are produced by the middle silk gland (MSG), and are a family of globular, water-soluble proteins which ensure the cohesion of the cocoon by sticking the fibroin fibres together. The sericins are characterized by their high serine content, where serine represents at least about 20 mol % of the total amino acid residues. Typically serine represents from about 20 to about 40 mol %, preferably from about 30 to about 40 mol % of the total amino acid residues.

A peptide consisting of 38 amino acids has been identified as a highly conserved and internally repetitive sequence of B. mori silk sericins. The consensus sequence of this peptide (Ser-Ser-Thr-Gly-Ser-Ser-Ser-Asn-Thr-Asp-Ser-Asn-Ser-Asn-Ser-Ala-Gly-Ser-Ser-Thr-Ser-Gly-Gly-Ser-Ser-Thr-Tyr-Gly-Tyr-Ser-Ser-Asn-Ser-Arg-Asp-Gly-Ser-Val) is characterized by its similarity to the average amino acid composition of silk sericin and its high hydrophilic amino acid content.

Native B. mori silk sericins range in molecular weight from about 10 to about 400 kDa, depending on gene coding and post-translational modifications. Three major fractions of silk sericin have been isolated from the B. mori cocoon, with molecular weights of about 150, 250, and 400 kDa respectively.

Silk sericins for use in the invention may be naturally derived, typically by extraction from silkworm (e.g. B. mori) cocoons or by extraction from raw silk. During the extraction process, the silk sericin may be hydrolysed to a certain extent, depending on the extraction method, temperature, pH and processing time. Accordingly, the molecular weight which is quoted for such materials will represent the average molecular weight of the various protein, polypeptide, oligopeptide and amino acid constituents present. The average molecular weight of silk sericins derived by boiling water extraction of cocoons generally ranges from about 65 kDa to about 400 kDa. If an alkaline solution is used, such as one containing sodium hydroxide, the average molecular weight of the derived silk sericin generally ranges from about 1 kDa to about 50 kDa.

Silk sericins for use in the invention may also be artificially synthesized using conventionally known biological methods, for example, by inserting the silk sericin gene sequence into E. coli, in which the E. coli produces recombinant silk sericin, or by conventionally known chemical methods such as Fmoc/tBu solid-phase peptide synthesis.

Artificially synthesized silk sericins will generally contain several repeats of the 38 amino acid consensus sequence described above (Ser-Ser-Thr-Gly-Ser-Ser-Ser-Asn-Thr-Asp-Ser-Asn-Ser-Asn-Ser-Ala-Gly-Ser-Ser-Thr-Ser-Gly-Gly-Ser-Ser-Thr-Tyr-Gly-Tyr-Ser-Ser-Asn-Ser-Arg-Asp-Gly-Ser-Val), for example 2 to 8 repeats, more preferably 2 to 6 repeats.

Mixtures of any of the above described types of silk sericins may also be used in the invention.

Preferred silk sericins for use in the invention are naturally derived, and have an average molecular weight ranging from about 1 kDa to about 50 kDa, more preferably from about 5 to about 30 kDa and most preferably from about 10 kDa to about 30 kDa,

An example of a preferred silk sericin for use in the invention is silk sericin extracted from silkworm (e.g. B. mori) cocoons or raw silk, and containing serine at a level of from about 30 to about 40 mol % of the total amino acid residues, and having an average molecular weight ranging from about 1 kDa to about 50 kDa, more preferably from about 5 to about 30 kDa and most preferably from about 10 kDa to about 30 kDa.

In the hair shaping composition of the invention, the level of component (i) preferably ranges from 0.5 to 6%, more preferably from 1 to 3% and most preferably from 1.5 to 2.5% by weight based on the total weight of the composition.

The hair shaping composition of the invention comprises, as component (ii), one or more aliphatic di- or tricarboxylic acids or salts or hydrates thereof.

Aliphatic di- or tricarboxylic acids for use in the invention typically have a molecular weight (Mw) ranging from 60 to 300 g/mol and at least one pKa value (measured at 25° C. in water) ranging from 2.5 to 4.

Illustrative examples of dicarboxylic acids for use in the invention correspond to the following general formula:

HOOC—R¹—OOOH

in which R¹ is a divalent, saturated or unsaturated, linear or branched hydrocarbyl radical having from 1 to 4 carbon atoms, and which may optionally be substituted with one or more hydroxyl groups.

Preferably R¹ is a divalent saturated linear alkyl radical of formula —[CH(X)]ₙ— in which n is an integer ranging from 1 to 3 and each X is independently selected from —H and —OH.

Specific examples of such dicarboxylic acids include malonic acid and tartaric acid.

Illustrative examples of tricarboxylic acids for use in the invention correspond to the following general formula:

HOOC—CH₂—R²—COOH

in which R² is a divalent, saturated or unsaturated, linear or branched hydrocarbyl radical having from 1 to 3 carbon atoms, which is substituted with one —COOH group and which may optionally be substituted with one or more hydroxyl groups.

Specific examples of such tricarboxylic acids include citric acid, aconitic acid and tricarballylic acid.

Suitable salts include those with counterions such as alkali metal (preferably sodium), alkaline-earth metal (preferably calcium), ammonium and substituted ammonium ions.

Mixtures of any of the above-described aliphatic di- or tricarboxylic acids or salts or hydrates thereof may also be used.

More preferably component (ii) is selected from citric acid or salts or hydrates thereof, such as monosodium citrate, trisodium citrate, tricalcium citrate, trisodium citrate dihydrate, tripotassium citrate, monosodium citrate anhydrous, citric acid anhydrous, citric acid monohydrate and mixtures thereof.

Most preferably citric acid anhydrous and/or citric acid monohydrate is used as component (ii) in the hair shaping composition of the invention.

In the hair shaping composition of the invention, the level of component (ii) (preferably citric acid anhydrous and/or citric acid monohydrate) preferably ranges from 0.5 to 6%, more preferably from 0.5 to 5% and most preferably from 0.5 to 2.5% by weight based on the total weight of the composition.

In the hair shaping composition of the invention, the weight ratio of component (i) to component (ii) (preferably citric acid anhydrous and/or citric acid monohydrate) preferably ranges from about 8:1 to about 1:5, and more preferably ranges from about 4:1 to about 2:5.

Advantageously, the hair shaping composition of the invention does not require the incorporation of reducing agents, and a hair shaping composition according to the invention is generally substantially free of such materials.

The term “substantially free” in the context of this invention means that reducing agents are absent or included in trace quantities only, such as no more than 0.1%, preferably no more than 0.01%, and more preferably from 0 to 0.001% by weight based on the total weight of the composition.

The term “reducing agent” in the context of this invention means an agent which is effective to break hair disulfide bonds when applied to hair for a period ranging from about 3 to 15 minutes and at a temperature ranging from about 20 to 30° C. Examples of such reducing agents are ammonium thioglycolate (in a solution having a pH of between about 7 and 10.5), glyceryl monothioglycolate (employed at a pH of less than 7), thioglycolic acid, dithioglycolic acid, mercaptoethyl amine, mercaptopropionic acid, dithioglycolate and alkali metal or ammonium sulfites or bisulfites.

A hair shaping composition according to the invention may suitably have a conditioning gel phase, which may be generally characterized as a gel (Lβ) surfactant mesophase consisting of surfactant bilayers. Such a conditioning gel phase may be formed from a cationic surfactant, a high melting point fatty alcohol and an aqueous carrier. Typically these components are heated to form a mixture, which is cooled under shear to room temperature. The mixture undergoes a number of phase transitions during cooling, normally resulting in a gel (L_{β}) surfactant mesophase consisting of surfactant bilayers.

Examples of suitable cationic surfactants which are useful for forming the conditioning gel phase include quaternary ammonium cationic surfactants corresponding to the following general formula:

[N(R¹)(R²)(R³)(R⁴)]⁺(X)⁻

in which R¹, R², R³, and R⁴ are each independently selected from (a) an aliphatic group of from 1 to 22 carbon atoms, or

(b) an aromatic, alkoxy, polyoxyalkylene, alkylamido, hydroxyalkyl, aryl or alkylaryl group having up to 22 carbon atoms; and X is a salt-forming anion such as those selected from halide, (e.g. chloride, bromide), acetate, citrate, lactate, glycolate, phosphate nitrate, sulphate, and alkylsulphate radicals.

The aliphatic groups can contain, in addition to carbon and hydrogen atoms, ether linkages, and other groups such as amino groups. The longer chain aliphatic groups, e.g., those of about 12 carbons, or higher, can be saturated or unsaturated. Specific examples of such quaternary ammonium cationic surfactants of the above general formula are cetyltrimethylammonium chloride, behenyltrimethylammonium chloride (BTAC), cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, dipalmitoylethyldimethylammonium chloride, PEG-2 oleylammonium chloride and salts of these, where the chloride is replaced by other halide (e.g., bromide), acetate, citrate, lactate, glycolate, phosphate nitrate, sulphate, or alkylsulphate.

In a preferred class of cationic surfactant of the above general formula, R¹ is a C₁₆ to C₂₂ saturated or unsaturated, preferably saturated, alkyl chain and R², R³ and R⁴ are each independently selected from CH₃ and CH₂CH₂OH, preferably CH₃.

Specific examples of such preferred quaternary ammonium cationic surfactants for use in forming the conditioning gel phase are cetyltrimethylammonium chloride (CTAC), behenyltrimethylammonium chloride (BTAC) and mixtures thereof.

Mixtures of any of the above-described cationic surfactants may also be suitable.

The level of cationic surfactant suitably ranges from 0.1 to 10%, preferably from 0.2 to 5% and more preferably from 0.25 to 4% (by total weight of cationic surfactant based on the total weight of the composition).

By “high melting point” in the context of this invention is generally meant a melting point of 25° C. or higher. Generally the melting point ranges from 25° C. up to 90° C., preferably from 40° C. up to 70° C. and more preferably from 50° C. up to about 65° C.

The high melting point fatty alcohol can be used as a single compound or as a blend or mixture of at least two high melting point fatty alcohols. When a blend or mixture of fatty alcohols is used, the melting point means the melting point of the blend or mixture.

Suitable fatty alcohols of this type have the general formula R—OH, where R is an aliphatic carbon chain. Preferably R is a saturated aliphatic carbon chain comprising from 8 to 30 carbon atoms, more preferably from 14 to 30 carbon atoms and most preferably from 16 to 22 carbon atoms.

R can contain, in addition to carbon and hydrogen atoms, ether linkages, and other groups such as amino groups.

Most preferably, the fatty alcohol has the general formula CH₃(CH₂)ₙ OH, where n is an integer from 7 to 29, preferably from 15 to 21.

Specific examples of suitable fatty alcohols are cetyl alcohol, stearyl alcohol, behenyl alcohol, and mixtures thereof. Cetyl alcohol, stearyl alcohol and mixtures thereof are particularly preferred.

Mixtures of any of the above-described fatty alcohols may also be suitable.

The level of fatty alcohol suitably ranges from 0.01 to 10%, preferably from 0.1 to 8%, more preferably from 0.2 to 7% and most preferably from 0.3 to 6% (by weight based on the total weight of the composition).

The weight ratio of cationic surfactant to fatty alcohol is suitably from 1:1 to 1:10, preferably from 1:1.5 to 1:8, optimally from 1:2 to 1:5.

A hair shaping composition according to the invention may also incorporate other optional ingredients to enhance performance and/or consumer acceptability. Suitable optional ingredients include: preservatives, colouring agents, chelating agents, antioxidants, fragrances, antimicrobials, antidandruff agents, cationic conditioning polymers, styling ingredients, sunscreens, proteins and hydrolysed proteins.

The pH of the hair shaping composition of the invention is 6 or less, and preferably 4 or less. More preferably the pH of the hair shaping composition ranges from 1.5 to 3.8, most preferably from 2.5 to 3.5 and ideally from 2.8 to 3.2.

Preferably, the hair shaping composition is a single dose composition. The term “single dose” in the context of this invention means that the composition is to be topically applied to the hair in one go.

The hair shaping composition of the invention is suitable for topical application to hair for improved hair volume-down. The term “volume-down” in the context of this invention generally means reduced visible bulkiness of the hair. For many consumers, improved hair volume-down provides a number of associated benefits, such as improved straightness, smoothness, manageability and style retention.

The hair shaping composition of the invention is preferably topically applied to the hair at a temperature from 15 to 40° C., and more preferably at a temperature from 20 to 30° C.

Preferably, the composition is applied to dry hair. The term “dry hair” in the context of this invention generally means hair from which free water (i.e. water disposed as a film or droplets on the cuticle surface) has been substantially removed. Hair may be dried by exposure to air, by use of a heated hair drying appliance, by rubbing with a water-absorbent article, or by a combination of any of these methods. Preferably, the dry hair will not have been washed or actively wetted, (such as by shampooing, conditioning, rinsing or otherwise treating with an aqueous composition) in the preceding 2 hours and more preferably in the preceding 3 hours prior to topical application of the composition, and will have been permitted to acclimatise to atmospheric conditions. In such circumstances there is substantially no free water present which interferes with the adsorption of the composition on application. A suitable indicator of dry hair in the context of this invention would be a hair fibre whose calculated water content does not exceed 25% by weight based on the total weight of the hair fibre.

After topical application to the hair, it is preferred that the hair shaping composition is allowed to remain in contact with the hair without rinsing. More preferably, the hair shaping composition is allowed to remain in contact with the hair without rinsing until the hair thus treated is dry.

The hair thus treated may be dried naturally by exposure to air, by use of a heated hair drying appliance, by rubbing with a water-absorbent article, or by a combination of any of these methods.

The hair shaping composition may thus remain in contact with the hair after topical application for a period of at least about 3 minutes up to 3 hours or more if the hair is allowed to dry naturally.

In step (b) of the method of the invention, the treated hair is mechanically shaped.

Mechanical shaping of the hair in the method of the invention can be accomplished by such means as the finger tips, a plastic hair pick or the tail of a comb, the shaping being performed on portions of the hair comprising strands of hair in various numbers.

Using such means the hair may be pulled, combed, smoothed, pressed or flattened into a straightened configuration; or shaped gently into bends, waves or curls.

Preferably in step (b) of the method of the invention, the hair is mechanically shaped by mechanically straightening it. For example, the hair may be pulled, combed, smoothed, pressed or flattened into a straightened configuration.

A hot tool, such as an electrically heated flat hair iron or hand-held hair dryer, may be used in the mechanical shaping step. Such tools apply high levels of heat directly to the hair. Most operate in the 45° C. to 250° C. range, and are usually employed at temperature settings ranging from 50° C. to about 220° C., depending on the particular tool.

However, the present inventors have surprisingly found that the use of hot tools is not essential in the method of the invention. This is especially advantageous for consumers who wish to reduce or avoid the exposure of their hair to high temperatures, for example if their hair is fragile or overprocessed from previous chemical treatments such as bleaching and perming.

Accordingly the mechanical shaping of the hair in step (b) of the method of the invention is preferably conducted at a temperature from 15 to 40° C., such as from 20 to 30° C.

Most preferably in step (b) of the method of the invention the hair is mechanically straightened by combing it into a straightened configuration at a temperature from 15 to 40° C., such as from 20 to 30° C.

Surprisingly, the inventors have found that the improved “volume-down” provided by the hair shaping composition in accordance with the invention is capable of persisting after washing.

Accordingly the invention also provides a method for shaping and re-shaping hair comprising the following steps:
(a) treating the hair by topical application of a hair shaping composition as defined above;
(b) mechanically shaping the treated hair;
(c) rinsing the shaped hair, and
(d) mechanically re-shaping the rinsed hair.

In a typical method for shaping and re-shaping hair according to the invention, the hair shaping composition is topically applied to dry hair and the hair thus treated is combed straight at a temperature from 15 to 40° C., such as from 20 to 30° C. The treated, combed hair is dried (or allowed to dry) without rinsing the composition from the hair, and the dry hair is then mechanically straightened by combing it into a straightened configuration at a temperature from 15 to 40° C., such as from 20 to 30° C. The hair shaping composition is then rinsed from the hair at the next wash: typically after a period of about 24 to 72 hours following the initial application of the composition in step (a). The rinsed hair is then mechanically re-shaped.

The rinsing step may be conducted with water alone or with shampoo.

The use of hot tools is not essential in the re-shaping step. This is especially advantageous for consumers who wish to reduce or avoid the exposure of their hair to high temperatures, for example if their hair is fragile or overprocessed from previous chemical treatments such as bleaching and perming.

Accordingly the hair is preferably re-shaped by combing it into a straightened configuration at a temperature from 15 to 40° C., such as from 20 to 30° C.

Method steps (a) to (d) as described above may also be repeated over one or more (e.g. two, three or four) cycles.

The invention is further illustrated with reference to the following, non-limiting Examples.

In the Examples, all ingredients are expressed by weight percent of the total formulation, and as level of active ingredient. Comparative Examples (not according to the invention) are indicated by letter; Examples according to the invention are indicated by number.

### EXAMPLES

### Examples 1 to 3 and (Comparative) Examples A to D

Dark brown European wavy#6 switches of length 25 cm and weight 2 gms, were soaked for 30 minutes in the following test solutions:

Example A: Aqueous solution, 2% silk sericin*

Example B Aqueous solution, 0.5% citric acid

Example 1: Aqueous solution, 2% silk sericin* and 0.5% citric acid

Example C Aqueous solution, 2.5% citric acid

Example 2: Aqueous solution, 2% silk sericin* and 2.5% citric acid

Example D Aqueous solution, 5% citric acid

Example 3: Aqueous solution, 2% silk sericin* and 5% citric acid

Control switches were soaked in water.

All switches were then combed straight and naturally dried. When dry the switches were combed straight and pictures taken. The volumes of the switches were measured using an Image analysis kit. The switches were subsequently washed a number of times, combed straight, dried and pictures taken after combing. If the switches are visually straight then the volume of the switches (actually the projection of the volume in mm² onto an image plane) is a measure of the straightness benefits of the leave-on treatment

The results are shown in Table 1.

TABLE 1
Volumes of treated hair switches in mm² after a single treatment (no
heat) and subsequent four washes.

| | after initial | | after 2nd | | after 4th | |
|---|---|---|---|---|---|---|
| | treatment | | wash | | wash | |
| | | % | | % | | % |
| Treatment | volume | benefit | volume | benefit | volume | benefit |
| Control (water) | 14729 | 0.0 | 13599 | 0.0 | 14118 | 0.0 |
| Example A | 8007 | 45.6 | 12409 | 8.7 | 15261 | −8.1 |
| Example B | 9302 | 36.8 | 11131 | 18.2 | 11175 | 20.8 |
| Example 1 | 7032 | 52.3 | 10098 | 25.7 | 10530 | 25.4 |
| Example C | 8870 | 39.8 | 11144 | 18.1 | 11921 | 15.6 |
| Example 2 | 6401 | 56.5 | 8900 | 34.6 | 9375 | 33.6 |
| Example D | 7885 | 46.5 | 10600 | 22.0 | 10656 | 24.5 |
| Example 3 | 6346 | 56.9 | 9601 | 29.4 | 9671 | 31.5 |

From the table it can be seen that the compositions according to the invention with a combination of silk sericin and citric acid provide improved straightening compared to either silk sericin or citric acid. Furthermore, the combination according to the invention provides synergistic “after wash” straightening benefits, with treated switches maintaining a significant degree of straightness even after four washes.

### Example 4

The following formulation illustrates a hair shaping composition according to the invention

| Ingredient | % activity | % w/w raw material |
|---|---|---|
| Behentrimonium chloride | 70 | 1.1429 |
| Cetearyl alcohol | 100 | 3.0 |
| Perfume | 100 | 0.60 |
| Preservative | 100 | 0.2 |
| Dimethicone emulsion | 70 | 1.429 |
| Citric Acid | 100 | 2.5 |
| Silk sericin* | 100 | 2.0 |
| Water | 100 | To 100% |

*Sericin powder molecular weight 20 kDa, ex Huzhou Xintiansi Bio-tech Co., Ltd., China

1. A hair shaping composition suitable for topical application to hair, the composition having a pH of 6 or less and comprising, in an aqueous continuous phase:
(i) from 1 to 3% (by weight based on the total weight of the composition) of one or more silk sericins, and
(ii) from 0.5 to 6% (by weight based on the total weight of the composition) of one or more of citric acid anhydrous and/or citric acid monohydrate.

2. The composition according to claim 1, in which component (i) is silk sericin extracted from B. mori cocoons or raw silk, and containing serine at a level of from 30 to 40 mol % of the total amino acid residues, and having an average molecular weight ranging from 1 kDa to 50 kDa.

3. The composition according to claim 1, in which the average molecular weight of the silk sericin ranges from 10 kDa to 30 kDa.

4-5. (canceled)

6. The composition according to claim 1, in which the level of component (ii) ranges from 0.5 to 2.5% by weight based on the total weight of the composition.

7. The method for shaping hair which comprises the steps of (a) treating the hair by topical application of a hair shaping composition according to claim 1, followed by (b) mechanically shaping the treated hair.

8. The method according to claim 7, in which in step (b) the hair is mechanically straightened by combing it into a straightened configuration at a temperature from 15 to 40° C.

9. The method according to claim 7, in which the hair shaping composition is topically applied to dry hair.

10. The method for shaping and re-shaping hair comprising the following steps:
(a) treating the hair by topical application of a hair shaping composition according to claim 1;
(b) mechanically shaping the treated hair;
(c) rinsing the shaped hair, and
(d) mechanically re-shaping the rinsed hair.
